(19) United States
(12) Patent Application Publication
(10) Pub. No.: US 2010/0275924 A1
(43) Pub. Date: Nov. 4, 2010

(54) System for Removal of Pooled Secretions

(76) Inventors: Mark Zielke, Plano, TX (US); Rosanna Orlovsky, Plano, TX (US)

Correspondence Address: Mark Zielke;Rosanna Orlovsky, 2201 Grinelle Drive, Plano, TX, 75025, US

(21) Appl. No.: 12/536,536

(22) Filed: Aug. 6, 2009

Related U.S. Application Data
(60) Provisional application No. 61/175,125, filed on May 4, 2009.

(51) Int. Cl. A61M 1/00; A61M 16/04

(52) U.S. Cl. 128/207.14; 604/319

(57) ABSTRACT

A system for removal of pooled secretions is provided. The system includes one or more tubes, at least one reservoir coupled to the one or more tubes; a cap detachably coupled to the at least one reservoir; and a suction mechanism having a button and is coupled to the at least one reservoir.

### CROSS-REFERENCES TO RELATED APPLICATIONS

The present application claims the benefit of U.S. Provisional Application No. 61/175,125, entitled “SYSTEM FOR REMOVAL OF POOLED SECRETIONS,” filed on May 4, 2009, which is hereby incorporated by reference herein in its entirety.

### FIELD OF THE TECHNOLOGY

The present disclosure generally relates to closed systems for storing fluids and, more specifically, to removal of the stored fluids.

### DESCRIPTION OF THE RELATED ART

Healthcare is vital part of society. Healthcare providers are constantly developing methodologies and systems to improve healthcare in the United States. In particular, disease reduction and prevention at healthcare facilities among its patients is still a huge concern in the healthcare industry. For example, Ventilator Acquired Pneumonia (VAP) is a dangerous medical infection that results in patients who are on mechanical ventilation through an endotracheal or tracheotomy tube for at least 48 hours when pooled secretions are unintentionally poured back into a patient's endotracheal or tracheotomy tube. As such, it would be desirable to provide a closed system that removes pooled secretions from a patient.

### SUMMARY

In one aspect, a system for removal of pooled secretions is provided. The system includes one or more tubes, at least one reservoir coupled to the one or more tubes; a cap detachably coupled to the at least one reservoir; and a suction mechanism having a button and is coupled to the at least one reservoir.

In another aspect, a secretion removal device is provided. The secretion removal device includes one or more tubes; at least one reservoir coupled to the one or more tubes; a connector; a cap detachably coupled to the at least one reservoir; a suction mechanism having a button and is coupled to the at least one reservoir via the connector, and wherein the suction mechanism is configured to exert a suction force on the fluid in the reservoir such that the fluid is pulled out of the reservoir upon the pressing of the button.

In still another aspect, a means for removing pooled secretions. The means includes a means for transmitting one or more fluids; a means for storing the one or more fluids; a means for exerting a suction force on the one or more fluids; and a means for controlling the suction force exerted on the one or more fluids.

In yet another aspect, a method for removing pooled secretions. The method includes transmitting one or more fluids; storing the one or more fluids; exerting a suction force on the one or more fluids; and controlling the suction force exerted on the one or more fluids.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The present invention is described in detail below with reference to the attached drawing figures, which are incorporated by reference herein and wherein:

FIG. 1 is block diagram in accordance with an exemplary embodiment of the invention;

FIG. 2 is a schematic diagram of a connector in accordance with an exemplary embodiment of the invention; and

FIG. 3 is an aerial perspective of the connector in accordance with another exemplary embodiment of the invention; and

FIG. 4 is a flowchart in accordance with another exemplary embodiment of the invention.

### DETAILED DESCRIPTIONS

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

Broadly, an embodiment of the present invention generally provides a system that includes one or more tubes; at least one reservoir coupled to the one or more tubes; a cap detachably coupled to the at least one reservoir; and a suction mechanism having a button and is coupled to the at least one reservoir.

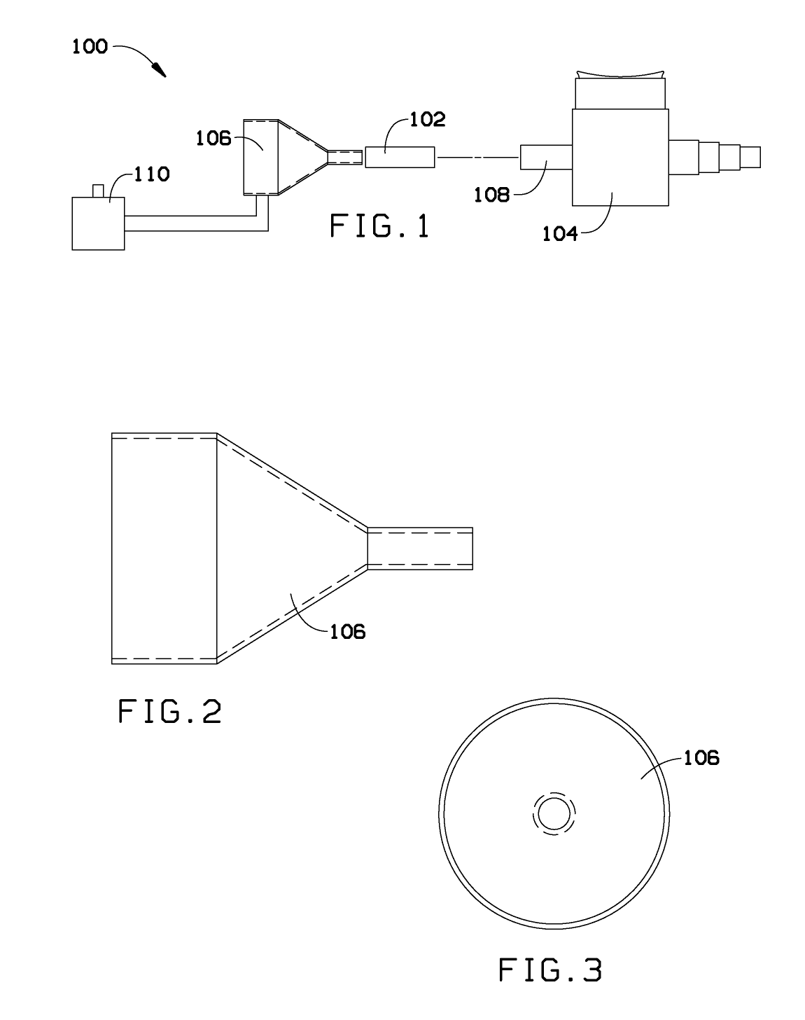

Embodiments of the present invention are discussed in connection with the following figures: FIGS. 1, 2, 3 and 4.

FIG. 1 shows a block diagram of a pooled secretion removal system. As shown in FIG. 1, the pooled secretion removal system 100 may include one or more tubes 102, at least one reservoir 104; a connector 106 (shown as a funnel); a cap 108 detachably coupled to the at least one reservoir 104; and a suction mechanism 110. The suction mechanism 110 may include a button (not shown). The suction mechanism 110 may be coupled to the at least one reservoir 104 via the connector 106. In one embodiment, the pooled secretion removal system 100 may be a closed system. Alternatively, the pooled secretion removal system 100 may be an open system or any system configuration known by one of ordinary skill in the art.

In some embodiments, the tubes 102 may be endotracheal tubes. In other embodiments, the tubes 102 may be tracheotomy tubes or any kind of tubes used by one of ordinary skill in the art. The tubes 102 may be made out of any material such as plastic, glass, or any material used by one of ordinary skill in the art. One example of a commonly used plastic in the art is polypropylene. The lengths and diameters of the tubes 102 may vary in accordance with the design choices known by those of ordinary skill in the art. For example, the tubes 102 may be designed to match the corresponding diameter of the connector 106. As such, the length may vary from 0.01 ml to 2 ml or any length desirable to one skilled in the art. Further, the diameter may vary from 0.01 mm to 3 mm or any diameter desirable to one skilled in the art.

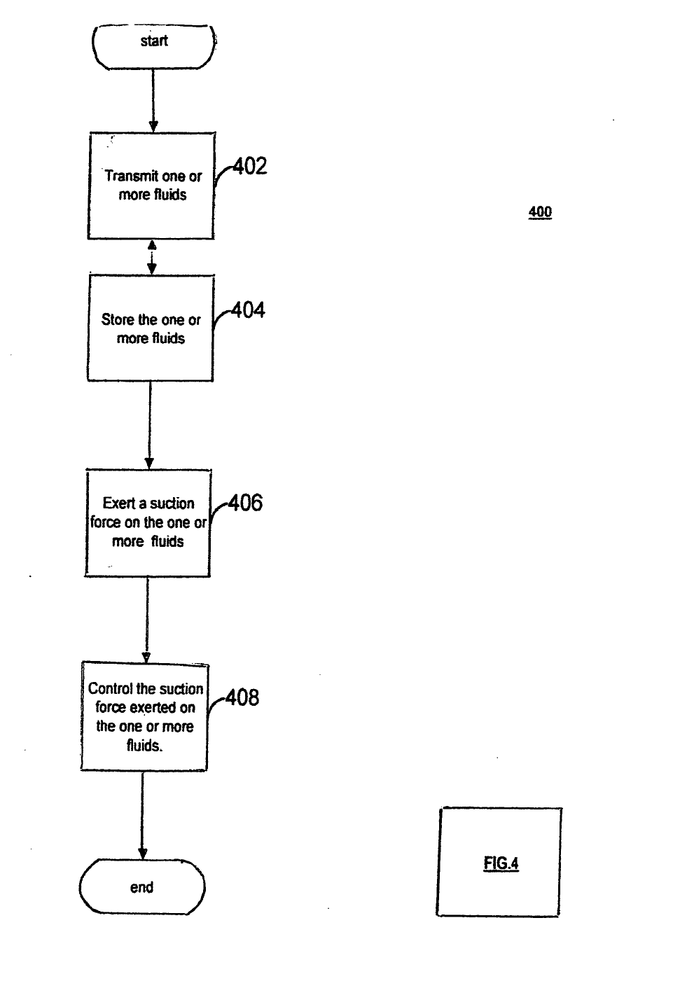

FIG. 2 shows a schematic diagram of the connector in accordance with an exemplary embodiment of the invention. As shown FIG. 2, the connector 106 is depicted as a funnel-shaped member. FIG. 3 shows an aerial perspective of the connector 106 in accordance with an embodiment of the invention.

The connector 106 may be any member capable of connecting at least two members of the pooled secretion removal system 100 such as funnel, tube, or any connecting member capable of passing fluid known by one of ordinary skill in the art. The connector 106 may be made out of any material such as plastic, glass, or any material used by one of ordinary skill in the art. The lengths and diameters of the connector 106 may vary in accordance with the design choices known by those of ordinary skill in the art. For example, the connector 106 may be designed to match the corresponding diameter of the connector 106. As such, the length may vary from 0.01 mm to 6 mm or any length desirable to one skilled in the art. Further, the diameter may vary from 0.01 mm to 22 mm or any diameter desirable to one skilled in the art. In some embodiments, the connector 106 may be a 22 mm diameter funnel-shaped connector approximately 3 mm long. Additionally, the connector 106 may be connected to a 1 ml long piece of suction tubing that may be connected to the suction mechanism 110.

The pooled secretion removal system 100 may be set up in the following manner. After setting up the ventilator in a health care facility (not shown) and a ventilator circuit also known as a “Ballard system” (not shown), a user may remove the end-cap of the ventilator circuit and replace it with the connector 106 coupled to the suction mechanism 110. Alternatively, the connector 106 coupled to the suction mechanism 110 may be connected to the wall suction. The connector 106 coupled to the suction mechanism 110 may remain in place for the duration of the patient's time on assisted ventilation. The connector 106 coupled to the suction mechanism 110 is completely removable for weaning purposes.

Alternatively, a health care professional may set up the pooled secretion removal system 100 by first placing the device snugly on the end of the “T” member of the ventilator circuit (not shown) where the end-cap was removed. The pooled secretion removal system 100 may then be ready to plug into the suction tubing coming from a wall suction canister.

The suction mechanism 110 may be coupled to the at least one reservoir 104 via the connector 106 or directly to ventilator circuit (not shown). The suction mechanism 110 may be configured to exert a suction force on the fluid in the reservoir 104 such that the fluid is pushed out of the reservoir upon the pressing of the button. When the health care professional notices secretions pooled at the end of the “T” piece, the health care professionals may unlock the suction device control of the suction mechanism 110, press down on the button to allow the suction to remove the secretions by means of plunger, stopper, or any suction means used by one of ordinary skill in the art. Once the secretions are cleared, the plunger is released thereby stopping the suction from the wall.

FIG. 4 shows a flowchart describing a process 400 in accordance with an exemplary embodiment of the invention (with regard to FIG. 1). At block 402, one or more fluids may be transmitted by one or more tubes. At block 404, the one or more fluids may be stored in at least one reservoir. At block 406, a suction force may be exerted on the one or more fluids via a suction mechanism. At block 408, the amount of suction force may be controlled by the suction mechanism that is exerted on the one or more fluids.

Although the forgoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

Thus, many modifications and variations may be made in the techniques and structures described and illustrated herein without departing from the spirit and scope of the present invention. Accordingly, it should be understood that the methods and apparatus described herein are illustrative only and are not limiting upon the scope of the invention.

What is claimed is:

1. A system for removal of pooled secretions, comprising:
one or more tubes;
at least one reservoir coupled to the one or more tubes;
a cap detachably coupled to the at least one reservoir; and
a suction mechanism having a button and is coupled to the at least one reservoir.

2. The system of claim 1, wherein the tubes are plastic tubes.

3. The system of claim 1, wherein the tubes are endotracheal tubes.

4. The system of claim 1, wherein the tubes are tracheotomy tubes.

5. The system of claim 1, further comprising: a connector for connecting at least two members.

6. The system of claim 5, wherein the connector is a funnel-shaped member.

7. A secretion removal device, comprising:
one or more tubes;
at least one reservoir coupled to the one or more tubes;
a connector;
a cap detachably coupled to the at least one reservoir;
a suction mechanism having a button and is coupled to the at least one reservoir via the connector, and
wherein the suction mechanism is configured to exert a suction force on the fluid in the reservoir such that the fluid is pulled out of the reservoir upon the pressing of the button.

8. The device of claim 7, wherein the tubes are plastic tubes.

9. The device of claim 7, wherein the tubes are endotracheal tubes.

10. The device of claim 7, wherein the tubes are tracheotomy tubes.

11. The device of claim 7, wherein the connector is a funnel-shaped member.

12. The device of claim 7, wherein the connector is configured to connect at least two members.

13. A means for removing pooled secretions, comprising:
a means for transmitting one or more fluids;
a means for storing the one or more fluids;
a means for exerting a suction force on the one or more fluids; and
a means for controlling the suction force exerted on the one or more fluids.

14. The means of claim 13, wherein the means for transmitting the fluids are plastic tubes.

15. The means of claim 13, wherein the means for transmitting the fluids are endotracheal tubes.

16. The means of claim 13, wherein the means for transmitting the fluids are tracheotomy tubes.

17. The means of claim 13, further comprising: a means for connecting at least two members.

18. The means of claim 13, wherein the means for connecting at least two members is a funnel-shaped member.

19. The system of claim 1, wherein the suction mechanism is configured to exert a suction force on the fluid in the reservoir such that the fluid is pulled out of the reservoir upon the pressing of the button.

20. The system of claim 1, wherein the suction mechanism is configured to be locked.
